(19) United States
(12) Patent Application Publication
(10) Pub. No.: US 2005/0109347 A1
(43) Pub. Date: May 26, 2005

(54) Surgical towel with x-ray detectable material

(76) Inventors: William Hallen Falls JR., Taipei (TW); Brian Terrance Mann, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/720,206

(22) Filed: Nov. 25, 2003

(51) Int. Cl. A61B 19/00

(52) U.S. Cl. 128/849

(57) ABSTRACT

A surgical towel with x-ray detectable material which eliminates the need for re-entry into a patent after operating in situations where operating towels are missing. The surgical towel comprises a sheet of fabric having x-ray detectable material sewn inside the hem woven into or otherwise attached to the fabric. The hem can be stitched with a thread of a different color than the color of the fabric to make the towel easily identifiable as being x-ray detectable. The x-ray detectable material can easily be observed by medical personnel upon examination of an x-ray. If no x-ray detectable material is observed, unnecessary re-entry into the patient is prevented.

### BACKGROUND OF THE INVENTION

1. Field of the Invention

The present invention relates to a surgical towel. More specifically, the present invention discloses a surgical towel, otherwise referred to as an operating towel, O.R. Towel or operating room towel, with x-ray detectable material which eliminates the need for re-entry into a patient after operating, to search for a missing towel, in situations where operating towels are, or may be, missing.

2. Description of the Prior Art

Prior to operating, medical staff prepare the patient for surgery by placing towels on the patient around the area to be operated on. This area is commonly known as the incision area. These towels are positioned so that they overlap and surround the area where the opening will be. Additionally, the towels may be placed on trays, and surgical tools can then be laid out on the towels.

The main purpose of the towels is to establish a clean and sanitary working area during surgery, and to contain smaller amounts of spilled or splashed blood. However, during the operation or surgery, after the incision is made by the surgeon, blood inevitably flows from the patient. Normally, laparotomy sponges, commonly referred to as “lap sponges”, made of gauze are used to absorb flowing blood and blood within the body. Unfortunately, in some situations, the blood flow is substantial, and the lap sponges are inadequate or unavailable. In these cases, medical personnel often use the towels for other than their intended purposes.

When this occurs, there is the possibility that the towel is placed inside the body. Since the towel is drenched in blood, it may become difficult to identify the towel. In some instances, towels have been left inside the body of the patient. If the towels are not noticed as missing, they can remain in the patient for some time before causing enough discomfort that the patient seeks medical help. This results in the patient having to undergo another surgery to search the operation site for a missing towel, and remove the towel if found.

However, it is currently extremely difficult to determine if a towel was left inside the patient without re-entry into the patient. As a result, patients may undergo unnecessary surgery for other issues, when in reality the cause is a misplaced towel.

Furthermore, not only has the patient unnecessarily suffered, but medical personnel are thus susceptible to malpractice lawsuits in these situations, which is extremely expensive.

Therefore, there is a need for an effective way of preventing unnecessary re-entry into a patient after surgery to determine whether a towel was left inside the patient, thereby reducing a patient's suffering and the risk of malpractice lawsuits.

### SUMMARY OF THE INVENTION

To achieve these and other advantages and in order to overcome the disadvantages of the conventional method in accordance with the purpose of the invention as embodied and broadly described herein, the present invention provides a surgical towel with x-ray detectable material which eliminates the need for re-entry into a patent after operating in situations where operating towels are missing or there is great patient distress following surgery and a missing object may be suspected.

Utilizing the towel of the present invention, the patient can easily be x-rayed to determine whether a towel has been left inside the patient after surgery.

Upon examination of the x-ray, the x-ray detectable material can easily be observed by medical personnel if a towel is still inside the patient. If no x-ray detectable material is observed, unnecessary re-entry into the patient is prevented.

The towels are used to cover Gurneys or operating beds and also provide a sterile site to lay sterilized instruments and operation tools on. The towels provide a sterile splash covering to absorb spilt or splashed blood, while also marking out an incision site. They are not typically intended to soak up blood like a gauze or lap sponge is.

An object of the present invention is to provide a surgical towel that comprises at least one piece of x-ray detectable material so that an x-ray will indicate the presence of a towel.

Another object of the present invention is to provide a surgical towel that comprises at least one piece of x-ray detectable material enclosed in a hem of the towel, woven into, ironed on, or any anyway attached to the towel, making manufacturing convenient and ensuring the x-ray detectable material is not inadvertently separated from the towel

Another object of the present invention is to provide a surgical towel with x-ray detectable material with the thread used to stitch the hem of a different color than the color of the towel to easily identify the towel as being x-ray detectable.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a drawing illustrating a surgical towel with x-ray detectable material according to an embodiment of the present invention; and

FIG. 2 is a drawing illustrating a surgical towel with x-ray detectable material according to an embodiment of the present invention.

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

The surgical towel of the present invention comprises a piece of fabric, for example, a woven single-ply sheet of 100% cotton. The fabric can also comprise synthetic material or be multi-ply. The fabric is dyed or undyed and be colored as desired. At least one edge of the fabric is folded over and stitched to create a hem. In an embodiment of the present invention, three sides of the towel are hemmed; however, one, two, three, or all four sides of the towel can be hemmed or un-hemmed.

The thread used for stitching the hem is for example, a synthetic thread. The color of the thread can be the same color as the towel; however, to add further advantages, the color of the thread can be a different color than the color of the towel. This allows the towel to be easily identified as being x-ray detectable.

Prior to stitching or during stitching, a piece of x-ray detectable material is placed inside the fold in the fabric in order to be enclosed in the hem. The x-ray detectable material is a piece of material that is easily detectable upon x-ray. In an embodiment of the present invention the x-ray detectable material comprises Barite, Barium, or BaSO4 and Polyvinyl Chloride (PVC). In an embodiment the material is a 60% BaSO4 and 40% PVC thread. The PVC material is provided to allow the thread to stretch and protects the BaSO4 material

The length or amount of x-ray material is selectable depending on cost, effectiveness, or convenience. Typically, a three to four inch piece is sufficient for detection.

Alternatively, the x-ray detectable material is a flexible strip which is sewn inside the hem or through which the hem stitching is sewn. Exposing a portion of the x-ray material allows the x-ray material to be seen without further identifying marks. Stitching through the material provides further adhesion of the x-ray detectable material to the towel.

Additionally, the x-ray detectable material can be shaped, coded, numbered, or have identifying characteristics. In situations where other x-ray detectable objects are intentionally placed inside a patient, the unique characteristics of the x-ray detectable material of the present invention easily identify the object as a surgical towel. For example, the material can have wording such as a brand name that can be read upon examination of an x-ray.

Furthermore, multiple pieces of the x-ray detectable material can be used to increase identification or detection. For example, a piece of material can be placed in several hems or locations of the towel.

Rather than enclosing the x-ray detectable material in the hem, the material can be sewn, woven into or adhered to the towel. Also, the x-ray detectable material can be attached to the towel so as to create a loop or tab. The loop adds the convenience of hanging the towel or easily grasped and the tab allows the towel to be easily grasped. Additionally, the material can be a label or inside a label attached to the towel. For example, a label with the manufacturer or brand name of the towel can comprise x-ray detectable material. This label indicates the towel maker and identifies the towel as being x-ray detectable.

Alternatively, the x-ray detectable material may also be woven into the towel as a line going through it, or in a manner that is readily identifiable in an x-ray. For example, the x-ray detectable material can be similar to a thread and stitched or woven into the towel.

Some towels are disposable but others are re-usable. The towels can be sterilized, for example by Gamma radiation or steam, or used unsterilized. Furthermore, the towels can be colorized to indicate whether they are disposable, re-usable, sterilized, or unsterilized.

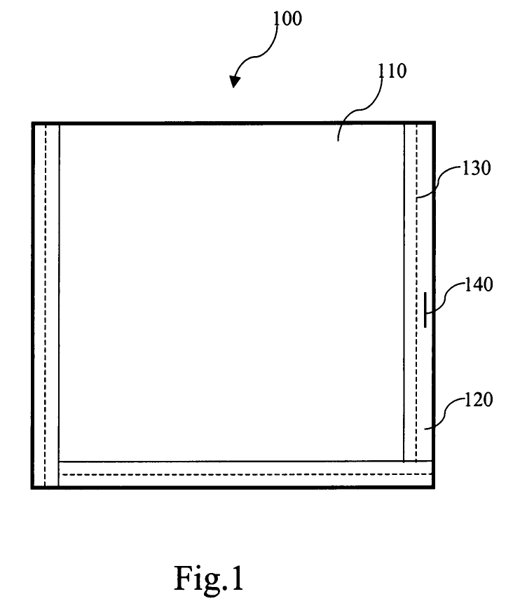

Refer to FIG. 1, which is a drawing illustrating a surgical towel with x-ray detectable material according to an embodiment of the present invention.

As shown in FIG. 1, the surgical towel 100 comprises a sheet of fabric 110, for example, a single-ply 100% cotton woven fabric. The towel 100 has at least one hem 120 stitched with thread 130. The towel 100 further comprises a piece of x-ray detectable material 140 enclosed in the hem 120. The color of the thread 130 used to stitch the hem 120 is a different color than the color of the fabric to make the towel 100 easily identifiable as having x-ray detectable material 140. Since the x-ray detectable material 140 is enclosed in the hem 120, the material 140 will remain attached to the towel 100. The towel 100 illustrated in FIG. 1 shows three sides of the fabric 110 having hems; however, any number of edges of the towel 100 can have hems.

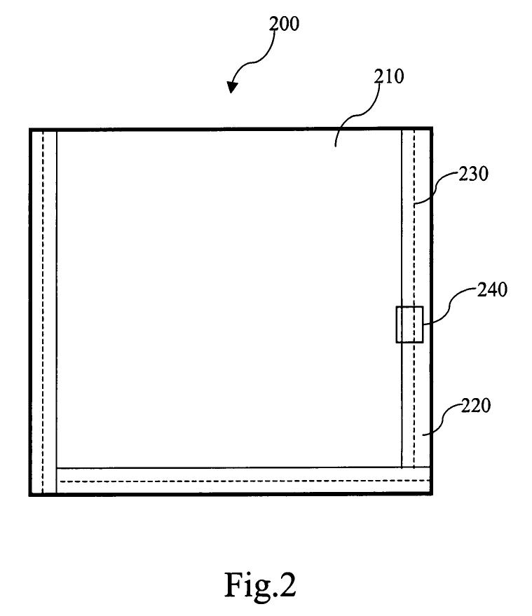

Refer to FIG. 2, which is a drawing illustrating a surgical towel with x-ray detectable material according to an embodiment of the present invention.

The towel 200 as shown in FIG. 2 is similar to the towel in FIG. 1. However, in this embodiment, the x-ray detectable material 240 is a flexible flat piece of material that is stitched over by the thread 230 and protrudes from the hem 220 of the fabric 210. Since the material 240 is exposed, the towel 200 is easily identifiable as being x-ray detectable. Additionally, the material 240 can have wording such as a manufacturer or brand name on the material 240.

As described above, the present invention provides a surgical towel with x-ray detectable material which eliminates the need for re-entry into a patent after operating in situations where operating towels are missing.

Utilizing the towel of the present invention, the patient can easily be x-rayed to determine whether a towel has been left inside the patient after surgery. Upon examination of the x-ray, the x-ray detectable material can easily be observed by medical personnel if a towel is still inside the patient. If no x-ray detectable material is observed, unnecessary re-entry into the patient is prevented thereby reducing a patient's suffering and the risk of malpractice lawsuits.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

1. A surgical towel, comprising:
a sheet of woven fabric comprising at least one stitched hem; and
at least one piece of x-ray detectable material enclosed in the hem, the x-ray detectable material allowing indication of the surgical towel upon x-ray.

2. The surgical towel of claim 1, whereby the at least one stitched hem is stitched with a thread of a different color than a color of the fabric.

3. The surgical towel of claim 1, whereby the piece of x-ray detectable material comprises Barite, Barium, or BaSO4.

4. The surgical towel of claim 3, whereby the piece of x-ray detectable material further comprises polyvinyl chloride (PVC).

5. The surgical towel of claim 4, whereby the piece of x-ray detectable material comprises 60% BaSO4 and 40% PVC.

6. The surgical towel of claim 1, whereby the sheet of woven fabric comprises cotton, synthetic material or a combination of these.

7. The surgical towel of claim 1, whereby the sheet of woven fabric is single-ply.

8. The surgical towel of claim 1, the x-ray detectable material comprising shaped, coded, numbered, or identifying characteristics to identify an x-rayed object as a surgical towel.

9. A surgical towel, comprising:
a sheet of single-ply woven cotton fabric having four edges;
at least one hem stitched in at least one of the four edges;
a thread for stitching the at least one hem; and
at least one piece of x-ray detectable material enclosed in one of the at least one hem.

10. The surgical towel of claim 9, whereby the thread is of a different color than a color of the fabric.

11. The surgical towel of claim 9, whereby the piece of x-ray detectable material comprises Barite, Barium, or BaSO4.

12. The surgical towel of claim 11, whereby the piece of x-ray detectable material further comprises polyvinyl chloride (PVC).

13. The surgical towel of claim 12, whereby the piece of x-ray detectable material comprises 60% BaSO4 and 40% PVC.

14. The surgical towel of claim 9, the x-ray detectable material comprising shaped, coded, numbered, or identifying characteristics to identify an x-rayed object as a surgical towel.

15. A surgical towel, comprising:
a sheet of fabric; and
at least one piece of x-ray detectable material attached to the sheet of fabric.

16. The surgical towel of claim 15, whereby the at least one piece of x-ray detectable material is attached to the sheet of fabric by being enclosed in a hem stitched in the sheet of fabric.

17. The surgical towel of claim 16, whereby the hem is stitched with a thread of a different color than a color of the fabric.

18. The surgical towel of claim 15, whereby the piece of x-ray detectable material comprises Barite, Barium, or BaSO4.

19. The surgical towel of claim 15, whereby the piece of x-ray detectable material is stitched or woven into or ironed on the sheet of fabric.

20. The surgical towel of claim 15, whereby the sheet of fabric comprises cotton, synthetic material or a combination of these.

21. The surgical towel of claim 15, whereby the sheet of fabric is single-ply.

22. The surgical towel of claim 15, the x-ray detectable material comprising shaped, coded, numbered, or identifying characteristics to identify an x-rayed object as a surgical towel.
